(19) United States
(12) Patent Application Publication
(10) Pub. No.: US 2008/0299628 A1
(43) Pub. Date: Dec. 4, 2008

(54) CONTINUOUS COUNTER-CURRENT ORGANOSOLV PROCESSING OF LIGNOCELLULOSIC FEEDSTOCKS

(75) Inventors: Christer Hallberg, Vancouver (CA); Donald O'Connor, Delta (CA); Michael Rushton, West Vancouver (CA); Edward Kendall Pye, Vancouver (CA); Gordon Gjennestadt, Vancouver (CA)

Correspondence Address: FASKEN MARTINEAU DUMOULIN, LLP, 2900 - 550 Burrard Street, VANCOUVER, BC, V6C 0A3, CA

(73) Assignee: Lignol Energy Corporation, Vancouver (CA)

(21) Appl. No.: 11/839,378

(22) Filed: Aug. 15, 2007

Related U.S. Application Data
(60) Provisional application No. 60/941,220, filed on May 31, 2007.

(51) Int. Cl. C12P 7/00; B01D 11/00; C12P 7/08; C13K 1/02; C12P 7/56; B01D 3/14

(52) U.S. Cl. 435/139; 127/1; 127/32; 127/37; 435/132; 435/140; 435/158; 435/165; 435/286.1; 435/289.1; 435/300.1

(57) ABSTRACT

A modular process for organosolv fractionation of lignocellulosic feedstocks into component parts and further processing of said component parts into at least fuel-grade ethanol and four classes of lignin derivatives. The modular process comprises a first processing module configured for physico-chemically digesting lignocellulosic feedstocks with an organic solvent thereby producing a cellulosic solids fraction and a liquid fraction, a second processing module configured for producing at least a fuel-grade ethanol and a first class of novel lignin derivatives from the cellulosic solids fraction, a third processing module configured for separating a second class and a third class of lignin derivatives from the liquid fraction and further processing the liquid fraction to produce a distillate and a stillage, a fourth processing module configured for separating a fourth class of lignin derivatives from the stillage and further processing the stillage to produce a sugar syrup.

### CROSS-REFERENCE TO RELATED APPLICATIONS

This application claims priority from our prior provisional application Ser. No. 60/941,220 filed May 31, 2007.

### FIELD OF THE INVENTION

This invention relates to fractionation of lignocellulosic feedstocks into component parts. More particularly, this invention relates to processes, systems and equipment configurations for recyclable organosolv fractionation of lignocellulosic material for continuous controllable and manipulable production and further processing of lignins, monosaccharides, oligosaccharides, polysaccharides and other products derived therefrom.

### BACKGROUND OF THE INVENTION

Industrial processes for production of cellulose-rich pulps from harvested wood are well-known and typically involve the steps of physical disruption of wood into smaller pieces and particles followed by chemical digestion under elevated temperatures and pressures to release and separate the cellulosic fibres from the constituent lignocellulosic fibrous matrices. The chemical digestion processes are commonly referred to as “kraft” and “sulfite” processes, and typically produce a solids fraction, referred to as pulp, comprising the cellulosic fibers and a liquids fraction commonly referred to as “black liquors” comprising the chemical solvents and solubilized materials released from the lignocellulosic fibrous matrices. The cellulosic fibrous pulps are typically used for paper manufacturing while the black liquors are usually processed to recover and recycle the chemical solvents, and the residues are typically combusted for in-house energy and/or heat production.

During the past two decades, those skilled in these arts have recognized that lignocellulosic materials including gymnosperm and angiosperm substrates (i.e., wood) as well as field crop and other herbaceous fibrous biomass, waste paper and wood containing products and the like, can be potentially fractionated using organic solvents for digestion, into multiple useful component parts that can be separated and further processed into high-value products such as fuel ethanol, lignins, furfural, acetic acid, purified monosaccharide sugars among others. Such systems have become known as “organosolv” and/or bio-refining systems (Pan et al., 2005, Biotechnol. Bioeng. 90: 473-481; Pan et al., 2006, Biotechnol Bioeng. 94: 851-861). Organosolv pulping processes and systems for lignocellulosic feedstocks are well-known and are exemplified by the disclosures in U.S. Pat. Nos. 4,941,944; 5,730,837; 6,179,958; and 6,228,177. After digestion has been completed in organosolv processes, the solids comprising the cellulosic fibrous pulps are separated from the spent digestion liquids i.e., black liquors and typically comprise organic solvents, solubilized lignins, soluble monosaccharides, oligosaccharides, polysaccharides, other organic compounds and minerals released from the wood during the chemical digestion. The black liquors are then usually processed to remove the soluble lignins after which, the organic solvents are recovered, purified and recycled. The lignins and remaining stillage from the black liquors are typically handled and disposed of as waste streams. Although it appears that biorefining using organosolv systems has considerable potential for large-scale fuel ethanol production, the currently available biorefining processes and systems are not yet economically attractive except at very large scale because they require expensive pretreatment steps and currently produce only low-value co-products (Pan et al., 2006, J. Agric. Food Chem. 54: 5806-5813).

### SUMMARY OF THE INVENTION

The exemplary embodiments of the present invention relate to systems, processes and equipment configurations for receiving and controllably commingling lignocellulosic feedstocks with counter-flowing organic solvents while providing suitable temperature and pressure conditions for fractionating the lignocellulosic feedstocks into component parts which are then subsequently separated. The separated component parts are further selectively, controllably and manipulably processed.

According to one exemplary embodiment of the present invention, there is provided a modular processing system for receiving therein and fractionating a lignocellulosic feedstock into component parts, separating the component parts into at least a solids fraction and a liquids fraction, and then separately processing the solids and liquids fractions to further produce useful products therefrom. Suitable modular processing systems of the present invention comprise at least:
a first module comprising a plurality of equipment configured for: (a) receiving and processing lignocellulosic fibrous feedstocks, then (b) commingling under controlled temperature and pressure conditions the processed feedstocks with suitable solvents configured for physico-chemically disrupting the lignocellulosic feedstock into a solids fraction comprising mostly cellulosic pulps and a liquid fraction comprising spent solvents containing therein at least lignins, lignin-related and/or lignin-derived compounds, monosaccharides, oligosaccharides and polysaccharides, dissolved and suspended solids comprising hemicelluloses and celluloses and other organic compounds, and (c) providing a first output stream comprising the solids fraction and a second output stream comprising the liquids fraction;
a second module comprising a plurality of equipment configured for: (d) receiving and controllably adjusting the viscosity of the solids fraction, (e) commingling the adjusted-viscosity solids fraction with suitable enzymes selected for hydrolysis and saccharification of the cellulosic pulps into a liquid stream comprising mostly monosaccharides but may also contain di-saccharides and tri-saccharides, (f) commingling the monosaccharides liquid stream with suitable fermenting microorganisms for production of an ethanol stream therefrom, (g) refining the ethanol to produce at least a fuel grade ethanol stream and de-alcoholized solvent stream, (h) further processing the de-alcoholized solvent-stillage stream to separate a first lignin fraction therefrom, and (i) recycling the de-lignified de-alcoholized solvent stream for controllably adjusting the viscosity of fresh solids fraction coming into the second module from the first output stream of the first module;
a third module comprising a plurality of equipment configured for (j) receiving the liquids fraction from the first module and separating a second lignin fraction therefrom thereby producing a first filtrate, (k) controllably intermixing the first filtrate with a supply of water or alternatively a suitable aqueous outputs stream from elsewhere in the third module, thereby precipitating a third lignin fraction therefrom, (i) separating the third lignin fraction from the diluted first filtrate thereby producing a second filtrate, (m) refining the second filtrate in a distillation tower thereby by capturing at least firstly, a portion of the suitable solvents commingled with the lignocellulosic feedstock in the first module, secondly, a furfural fraction, and thirdly, a stillage fraction, (n) controllably recharging the captured portion of the suitable solvents with a portion of the fuel ethanol produced in the second module; and
an optional fourth module comprising a plurality of equipment configured for receiving the stillage fraction from the third module and separating therefrom at least acetic acid-containing condensate, sugar syrups, a fourth lignin fraction, and a semi-solid/solid waste material.

According to one aspect, the plurality of equipment in the first module is configured to continuously receive and convey therethrough in one direction a lignocellulosic feedstock ending with the discharge of a cellulosic solids fraction, while concurrently counter-flowing a selected suitable solvent through the equipment in an opposite direction to the conveyance of the lignocellulosic feedstock ending in a discharge of a spent solvents liquid fraction.

According to another aspect, the plurality of equipment in the first module is configured to receive a batch of a lignocellulosic feedstock and to continuously cycle therethrough a selected suitable solvent therethrough until a suitable solids fraction is produced from the batch of lignocellulosic feedstock.

According to yet another aspect, the plurality of equipment in the second module is configured to sequentially: (a) receive and reduce the viscosity of the cellulosic solids fraction discharged from the first module, then (b) progressively hydrolyze and saccharify the cellulosic solids into suspended solids, dissolved solids, hemicelluloses, polysaccharides, oligosaccharides thereby producing a liquid stream primarily comprising monosaccharides, (c) ferment the liquid stream, (d) distill and refine the fermentation beer to separate the beer into at least a fuel-grade ethanol and a stillage stream, (e) de-lignify the stillage stream, and (f) recycle the de-lignified stillage stream for reducing the viscosity of fresh incoming cellulosic solids fraction discharged from the first module.

According to a further aspect, the plurality of equipment in the second module may be optionally configured to sequentially: (a) receive and reduce the viscosity of the cellulosic solids fraction discharged from the first module, then (b) concurrently hydrolyze and saccharify the cellulosic solids into monosaccharides while fermenting the monosaccharides in the same vessel, (c) distill and refine the fermentation beer to separate the beer into at least a fuel-grade ethanol and a stillage stream, (d) de-lignify the stillage stream, and (f) recycle the de-lignified stillage stream for reducing the viscosity of fresh incoming cellulosic solids fraction discharged from the first module.

According to another aspect, the modular processing system of the present system may be additionally provided with a fifth module comprising an anaerobic digestion system provided with a plurality of equipment configured for receiving the semi-solid/solid waste material from the fourth module, then liquifying and gasifying the waste material for the bio-production of methane, carbon dioxide and water.

According to another exemplary embodiment of the present invention, there is provided processes for fractionating a lignocellulosic feedstock into component parts. First, foreign materials exemplified by gravel and metal objects are separated using suitable means, from the incoming lignocellulosic material. An exemplary separating means is screening. If so desired, the screened lignocellulosic feedstock may be further screened to remove fines and over-size materials. Second, the screened lignocellulosic feedstock is controllably heated for example by steaming after which, the heated lignocellulosic feedstock is de-watered and then pressurized. Third, the heated and de-watered lignocellulosic feedstock is commingled and then impregnated with a suitable aqueous solvent. Fourth, the commingled lignocellulosic feedstock and organic solvent are controllably cooked within a controllably pressurized and temperature-controlled system for a selected period of time. During the cooking process, lignins and lignin-related compounds contained within the commingled and impregnated lignocellulosic feedstock will be dissolved into the organic solvent resulting in the cellulosic fibrous materials adhered thereto and therewith to disassociate and to separate from each other. The cooking process will also release monosaccharides, oligosaccharides and polysaccharides and other organic compounds for example acetic acid, in solute and particulate forms, from the lignocellulosic materials into the organic solvents. Those skilled in these arts refer to such organic solvents containing therein lignins, lignin-related compounds, monosaccharides, oligosaccharides and polysaccarides and other organic compounds, as “black liquors” or “spent liquors”.

According to one aspect, controllably counter-flowing the organic solvent against the incoming lignocellulosic feedstock during the cooking causes turbulence that facilitates and speeds the dissolution and disassociation of the lignins and lignin-related components from the lignocellulosic feedstock. However, it is within the scope of this invention to alternatively provide turbulence during the cooking process with a controllable flow of organic solvent directed in the same direction as the flow of lignocellulosic feedstock, i.e., a concurrent flow, thereby controllably intermixing the solvent and lignocellulosic feedstock together. It is also within the scope of this invention to controllably partially remove the organic solvent during the cooking process and to replace it with fresh organic solvent.

According to another aspect, the lignocellulosic feedstock may comprise at least one of physically disrupted angiosperm, gymnosperm, and field crop fibrous biomass segments exemplified by chips, saw dust, chunks, shreds and the like. It is within the scope of this invention to provide mixtures of physically disrupted angiosperm, gymnosperm, and field crop fibrous biomass segments.

According to yet another aspect, the lignocellulosic feed stock may comprise at least one of waste paper, wood scraps, comminuted wood materials, wood composites and the like. It is within the scope of this invention to intermix lignocellulosic fibrous biomass materials with one or more of waste paper, wood scraps, comminuted wood materials, wood composites and the like.

According to a further aspect, the liquor to wood ratio, operating temperature, solvent concentration and reaction time may be controllably and selectively adjusted to produce pulps and/or lignins having selectable target physico-chemical properties and characteristics.

According to another exemplary embodiment of the present invention, there are provided processes and systems for separating the disassociated cellulosic fibers i.e. pulp, from the black liquors, and for further and separately processing the pulp and the black liquors. The separation of pulp and black liquors may be done while the materials are still pressurized from the cooking process or alternatively, pressure may be reduced to about ambient pressure after which the pulp and black liquors are separated.

According to one aspect, the cellulosic fibrous pulp is recoverable for use in paper-making and other such processes.

According to another aspect, there are provided processes and systems for further selectively and controllably processing the cellulosic pulps produced as disclosed herein. The pH and/or the consistency of the recovered pulp may be adjusted as suitable to facilitate the hydrolysis of celluloses to monosaccharides, i.e., glucose moieties in hydrolysate solutions. Exemplary suitable hydrolysis means include enzymatic, microbial, chemical hydrolysis and combinations thereof.

According to yet another aspect, there are provided processes and systems for producing ethanol from the monosaccarides hydrolyzed from the cellulosic fibrous pulp, by fermentation of the hydrolysate solutions. It is within the scope of this invention to controllably provide inocula comprising one or more selected suitable strains from yeast species, fungal species and bacterial species, to facilitate and enhance the rates of fermentation and/or fermentation efficiencies and/or fermentation yields. Suitable yeasts are exemplified by Saccharomyces spp. and Pichia spp. Suitable Saccharomyces spp are exemplified by S. cerevisiae such as strains Sc Y1528, Tembec-1 and the like. Suitable fungal species are exemplified by Aspergillus spp. and Trichoderma spp. Suitable bacteria are exemplified by Escherichia coli, Zymomonas spp., Clostridium spp. and Corynebacterium spp. among others, naturally occurring and genetically modified.

According to a further aspect, there are provided processes and systems for concurrently saccharifying and fermenting the cellulosic pulps produced as disclosed herein. It is within the scope of the present invention to controllably hydrolyze the cellulosic fibrous pulps into monosaccharides by providing suitable hydrolysis means exemplified by enzymatic, microbial, chemical hydrolysis and combinations thereof, while concurrently and controllably fermenting the monosaccharide moieties produced therein. It is within the scope of this invention to controllably provide inocula comprising one or more selected strains of Saccharomyces spp. to facilitate and enhance the rates of concurrent fermentation and/or fermentation efficiencies and/or fermentation yields.

According to a further aspect, there are provided processes and systems for further processing the ethanol produced from the fermentation of the hydrolysate solutions. Exemplary processes include concentrating and purifying the ethanol by distillation, and de-watering or dehydration by passing the ethanol through at least one molecular sieve or alternatively, through a suitable membrane filtration system.

According to a further exemplary embodiment of the present invention, there are provided processes and systems for recovering lignins and lignin-related compounds from the black liquors. An exemplary process comprises cooling the black liquor immediately after separation from the cellulosic fibrous pulp, in a plurality of stages wherein each stage, heat is recovered with suitable heat-exchange devices and organic solvent is recovered using suitable solvent recovery apparatus as exemplified by evaporation and cooling devices. The stillage, i.e., the cooled black liquors from which at least some organic solvent has been recovered, are then further cooled, pH adjusted (e.g., increasing acidity) and then rapidly diluted with water to precipitate lignins and lignin-related compounds from the stillage. The precipitated lignins and lignin-related compounds are subsequently washed at least once and then dried.

According to one aspect, the de-lignified stillage is processed through a distillation tower to evaporate remaining organic solvent, and to concurrently separate and concentrate furfural. The remaining stillage is removed from the bottom of the distillation tower. It is within the scope of the present invention to optionally divert at least a portion of the de-lignified stillage from the distillation tower input stream into the ethanol production stream for producing ethanol therefrom. Alternatively or optionally, at least a portion of the remaining stillage removed from the bottom of the distillation tower may be diverted into the ethanol production stream for producing ethanol therefrom.

According another aspect, the stillage recovered from the bottom of the solvent recovery column, is further processed by: (a) decanting to recover complex organic extractives as exemplified by phytosterols, oils and the like, and then (b) evaporating the decanted stillage to produce (c) a stillage evaporate/condensate comprising acetic acid, and (d) a stillage syrup containing therein dissolved monosaccharides. The stillage syrup may be decanted to recover (e) novel previously unknown low molecular weight lignins. The decanted stillage syrup may be optionally evaporated to recover dissolved sugars.

It is within the scope of this invention to further process the recovered organic solvent by purification and concentration steps to make the recovered organic solvent useful for recycling back into continuous incoming lignocellulosic feedstock.

According to one aspect, an organic solvent is intermixed and commingled with the lignocellulosic feedstock for a selected period of time to pre-treat the lignocellulosic feedstock prior to commingling and impregnation with the counter-flowing (or alternatively, concurrently flowing) organic solvent.

### BRIEF DESCRIPTION OF THE DRAWINGS

The present invention will be described in conjunction with reference to the following drawings, in which:

FIG. 1 is a schematic flowchart of an exemplary embodiment of the present invention of a modular continuous counter-flow system for processing a lignocellulosic feedstock;

FIG. 2 is a schematic flowchart of the system from FIG. 1 additionally provided with a device for optionally diverting the sugar output stream to (a) the fuel ethanol production module, and (b) an anaerobic digestion module;

FIG. 3 is schematic flowchart showing an alternative configuration of the fuel ethanol production module for concurrent saccharification and fermentation processes within a single vessel;

FIG. 4 is a schematic flowchart of an exemplary anaerobic digestion module suitable for cooperating with the modular continuous counter-flow system of the present invention for processing a lignocellulosic feedstock;

FIG. 5 is a schematic flowchart of a continuous counter-flow processing system of the process re-configured into a batch through-put system; and

FIG. 6 is a schematic flowchart showing an alternative configuration for the batch through-put system shown in FIG. 5.

### DETAILED DESCRIPTION OF THE INVENTION

Exemplary embodiments of the present invention relate to systems, processes and equipment configurations for receiving and controllably commingling lignocellulosic feedstocks with counter-flowing aqueous organic solvents, thereby fractionating the lignocellulosic feedstocks into component parts which are then subsequently separated. The separated component parts are further selectively, controllably and manipulably processed. The exemplary embodiments of the present invention are particularly suitable for separating out from lignocellulosic feedstocks at least four structurally distinct classes of lignin component parts with each class comprising multiple derivative lignin compounds, while concurrently providing processes for converting other component parts into at least fuel-grade ethanol, furfurals, and monosaccharide sugar streams.

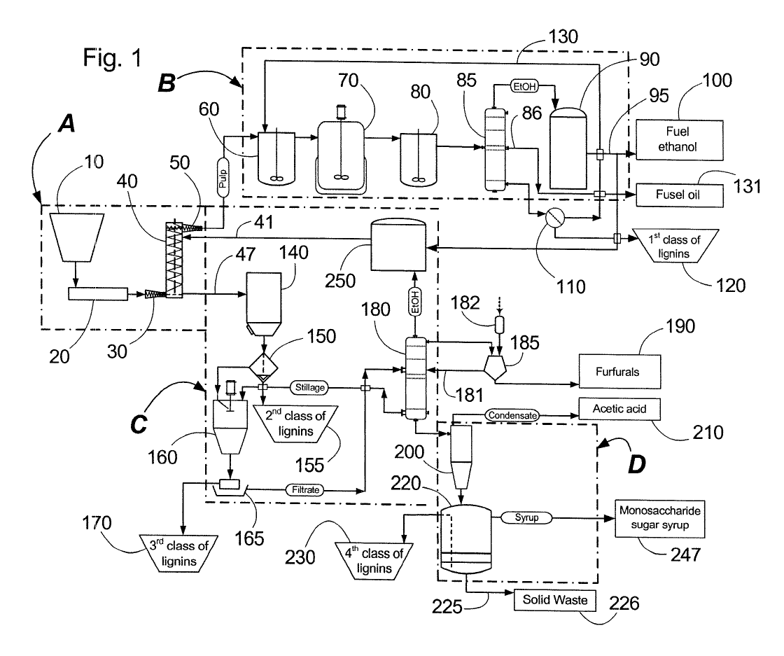

An exemplary modular processing system of the present invention is shown in FIG. 1 and generally comprises four modules A-D wherein the first module A is configured for receiving and processing lignocellulosic feedstocks into a solids fraction and a liquids fraction, the second module B is configured for receiving the solids fraction discharged from the first module A and producing therefrom at least a fuel ethanol output stream 100 and a first class of lignin derivatives 120 referred to hereafter as a very high molecular weight lignin (i.e., VHMW lignin), the third module C is configured for receiving the liquid fraction from the first module A and separating out at least a second class of lignin derivatives 155 referred to herein after as high molecular weight lignins (HMW lignins) and a third class of lignin derivatives 170 referred to hereafter as medium molecular weight lignins (i.e., MMW lignins), after which the filtrate is separated into at least recyclable distilled solvent, furfurals 190, and a stillage, and the fourth module D is configured for receiving and separating the stillage from the third module C into at least acetic acid 210, a fourth class of lignin derivatives 230 referred to hereafter as low molecular weight lignins (i.e., LMW lignins), a sugar syrup stream 247, and a semi-solid/solid waste material 226.

The first module A as exemplified in FIG. 1 is provided with a bin 10 configured for receiving and temporarily storing lignocellulosic feedstocks while continually discharging the feedstock into a conveyance system provided with a separating device 20 configured for removing pebbles, gravel, metal objects and other debris. A suitable separating device is a screening apparatus. The separating device 20 may be optionally configured for sizing the lignocellulosic feedstock into desired fractions. The processed lignocellulosic feedstock is then conveyed with a first auger feeder 30 into a first end of a digestion/extraction vessel 40 and then towards the second end of the digestion/extraction vessel 40. The vessel 40 is provided with an inlet approximate the second end for receiving a pressurized stream of a suitable heated digestion/extraction solvent which then counter-flows against the movement of the lignocellulosic feedstock through the vessel 40 thereby providing turbulence and commingling of the solvent with the feedstock. Alternatively, the inlet for receiving the pressurized stream of heated digestion/extraction solvent may be provided about the first end of the digestion/extraction vessel 40 or further alternatively, interposed the first and second ends of the digestion/extraction vessel 40. It is suitable to use aqueous organic solvents for the processes of the present invention. Exemplary suitable aqueous organic solvents include methanol, ethanol, propanol, butanol and the like. If so desired, the aqueous organic solvents may be additionally controllably acidified with an inorganic or organic acid. The vessel 40 is controllably pressurized and temperature controlled to enable manipulation of pressure and temperature so that target cooking conditions are provided while the solvent is commingling with the feedstock. Exemplary cooking conditions include pressures in the range of about 15-30 bar(g), temperatures in the range of about 120°-350° C., and pHs in the range of about 1.5-5.5. During the cooking process, lignins and lignin-related compounds contained within the commingled and impregnated lignocellulosic feedstock will be dissolved into the aqueous organic solvent resulting in the cellulosic fibrous materials previously adhered thereto and therewith to disassociate and to separate from each other. Those skilled in these arts will understand that in addition to the dissolution of lignins and lignin-related polymers, the cooking process will release monosaccharides, oligosaccharides and polysaccharides and other organic compounds for example acetic acid, in solute and particulate forms, from the lignocellulosic materials into the organic solvents. Those skilled in these arts refer to such organic solvents containing the lignins, lignin-related compounds, monosaccharides, oligosaccharides, polysaccharides, hemicelluloses and other organic compounds extracted from the lignocellulosic feedstock, as “black liquors” or “spent liquors”. The disassociated cellulosic fibrous materials released from the feedstock are conveyed to the second end of the vessel 40 where they are discharged via a second auger feeder 50 which compresses the cellulosic fibrous materials into a solids fraction, i.e., a pulp which is then conveyed to the second module B. The black liquors are discharged as a liquid fraction from about the first end of the digestion/extraction vessel 40 into a pipeline 47 for conveyance to the third module C.

The second module B is provided with a mixing vessel 60 wherein the viscosity of solids fraction, i.e., pulp discharged from the first module A is controllably reduced to a selected target viscosity, by commingling with a recovered recycled solvent stream delivered by a pipeline 130 from a down-stream component of module B. The reduced viscosity pulp is then transferred to a digestion vessel 70 where a suitable enzymatic preparation is intermixed and commingled with the pulp for progressively breaking down the cellulosic fibers, suspended solids and dissolved solids into hemicelluloses, polysaccharides, oligosaccharides and monosaccharides. A liquid stream comprising these digestion products is transferred from the digestion vessel 70 to a fermentation vessel 80 and is commingled with a suitable microbial inocula selected for fermentation of hexose and pentose monosaccharides in the liquid stream thereby producing a fermentation beer comprising at least a short-chain alcohol exemplified by ethanol and residual sediments. The fermentation beer is transferred to a first distillation tower 85 for refining by volatilizing then distilling and separately collecting from the top of the distillation tower 85 at least a fuel-grade ethanol which is transferred to a holding tank 90 and stored in a suitable holding container 100. We have discovered that the cellulosic solids fraction produced and separated in the first module A and delivered to the second module B for saccharification and fermentation, contains a unique class of lignin derivatives characterized by very high molecular weights (i.e., VHMW) in comparison to the classes of lignins commonly known to those skilled in these arts. The VHMW lignins are recoverable from the stillage produced in the second module B by removing stillage from the bottom of distillation tower 85 to separation equipment 110 configured to separate out the VHMW lignins which are then collected and stored in a suitable vessel 120 for further processing and/or shipment. It is within the scope of the present invention to heat the stillage to facilitate precipitation of the VHMW lignins prior to flowing the stillage through separation equipment 110. The de-lignified stillage may then be controllably recycled from equipment 110 via pipeline 130 to the mixing vessel 60 for reducing the viscosity of fresh incoming pulp from the first module A. Those skilled in these arts will understand that fusel oils comprising heavier alcohols exemplified amyl alcohols and furfurals are commonly produced in most fermentation processes and become concentrated in the “tails” and stillage at the ends of distillation runs. If allowed to accumulate in recycled stillage, the increasing concentrations of fusel oils will interfere with the fermentation efficiencies and rates. Therefore, as shown in FIGS. 1, 2, and 5, it is suitable to provide a bleed stream 86 communicating with a side draft on the first distillation tower 85 to enable periodic removal of the fusel oils to suitable storage vessels 131 prior to packaging and shipment for incorporation into other processes or alternatively, suitable disposal.

Suitable enzyme preparations for addition to digestion vessel 70 for progressively breaking down cellulosic fibers into hemicelluloses, polysaccharides, oligosaccharides and monosaccharides may comprise one or more of enzymes exemplified by cellulases, hemicellulases, β-glucosidases, β-xylosidases xylanases, α-amylases, β-amylases, pullulases and the like. Suitable microbial inocula for fermenting pentose and/or hexose monosaccharides in fermentation vessel 80 may comprise one or more suitable strains selected from yeast species, fungal species and bacterial species. Suitable yeasts are exemplified by Saccharomyces spp. and Pichia spp. Suitable Saccharomyces spp are exemplified by S. cerevisiae such as strains Sc Y1528, Tembec-1 and the like. Suitable fungal species are exemplified by Aspergillus spp. and Trichoderma spp. Suitable bacteria are exemplified by Escherichia coli, Zymomonas spp., Clostridium spp. and Corynebacterium spp. among others, naturally occurring and genetically modified. It is within the scope of the present invention to provide an inoculum comprising a single strain, or alternatively a plurality of strains from a single type of organism, or further alternatively, mixtures of strains comprising strains from multiple species and microbial types (i.e. yeasts, fungi and bacteria).

The black liquors discharged as a liquid fraction from the digestion/extraction vessel 40 of first module A, are processed in third module C to recover at least a portion of the digestion/extraction solvent comprising the black liquors, and to separate useful components extracted from the lignocellulosic feedstocks as will be described in more detail below. The black liquors are transferred by pipeline 47 into a flashing tower 140 wherein the pressure is reduced and the temperature subsequently reduced (i.e., “flashed”). We have discovered a second new class of lignin derivatives that precipitate from the liquids fraction during the flashing process that is characterized by high molecular weights (i.e., HMW) in comparison to the classes of lignins commonly known to those skilled in these arts. The HMW lignins 155 can be removed by a suitable separation device exemplified by a filter 150 thereby producing a first filtrate. The first filtrate is transferred to a mixing tank 160 where it is commingled with a supply of cold water thereby precipitating a third class of lignin derivatives characterized by medium molecular weights (MMW) from the first filtrate. Alternatively, a chilled stillage feed from a second distillation tower 180 may be used for commingling and intermixing with the first filtrate in the mixing tank 160 for precipitation of MMW lignins. MMW lignins are well-known and characterized. The precipitated MMW lignins are separated from the first filtrate by a suitable solids-liquids separation equipment 165 as exemplified by filtering apparatus, hydrocyclone separators, centrifuges and other such equipment, thereby producing a second filtrate. The separated MMW lignins are transferred to a lignin drier (not shown) for controlled removal of excess moisture, after which the dried MMW lignins are transferred to a storage bin 170 for packaging and shipping.

The second filtrate fraction is transferred from the separation equipment 165 to a second distillation tower 180 for vaporizing, distilling and recovering therefrom a short-chain alcohol exemplified by ethanol. The recovered short-chain alcohol is transferred to a digestion/extraction solvent holding tank 250 where it may, if so desired, be commingled with a portion of fuel-grade ethanol produced in module B and drawn from pipeline 95, to controllably adjust the concentration and composition of the digestion/extraction solvent prior to supplying the digestion/extraction solvent via pipeline 41 to the digestion/extraction vessel 40 of module A. It is within the scope of the present invention to recover furfurals from the de-lignified filtrate fraction concurrent with the vaporization and distillation processes within the second distillation tower, and transfer the recovered furfural rich side draw to a storage tank 190. An exemplary suitable process for recovering furfurals is to commingle the side drawl 81 with a water supply 182 and controllably cool or alternatively heat the commingled side draw to a suitable temperature thereby causing the side draw to separate into two phases; a lower oily furfural-rich phase and an upper aqueous ethanol phase. These two phases can be separated in a decanter 185, with the upper layer being returned to the distillation tower 180 via filtrate line 181 while the furfural-rich phase is transferred to a holding tank 190.

The stillage from the second distillation tower 180 is transferred to the fourth module D for further processing and separation of useful products therefrom. The hot stillage is transferred into a cooling tower or alternatively an evaporator 200 configured to collect a condensate comprising acetic acid that is then transferred to a suitable holding vessel 210. The stillage is then transferred to a stillage processing vessel 220 configured for heating the stillage thereby condensing and concentrating an oily layer at the bottom of the stillage. The oily layer comprises a fourth class of lignin derivatives well-known to those skilled in these arts, characterized by low molecular weights (LMW) which are then separated from a sugar syrup stream, and a semi-solid/solid waste material discharged into a waste disposal bin 226. The LMW lignins are transferred to a suitable holding container 230 for further processing and/or shipment. The sugar syrup stream, typically comprising at least glucose, mannose and galactose, is transferred to a suitable holding tank 247 prior to further processing and/or shipping. Those skilled in these arts will understand that the sugar syrup stream may be optionally diverted into a sixth optional module (not shown) comprising at least a fermentation vessel communicating with a distillation tower and stillage recovery equipment (not shown) for production of “sugar platform” chemicals exemplified by 1,3 propanediol, lactic acid and the like.

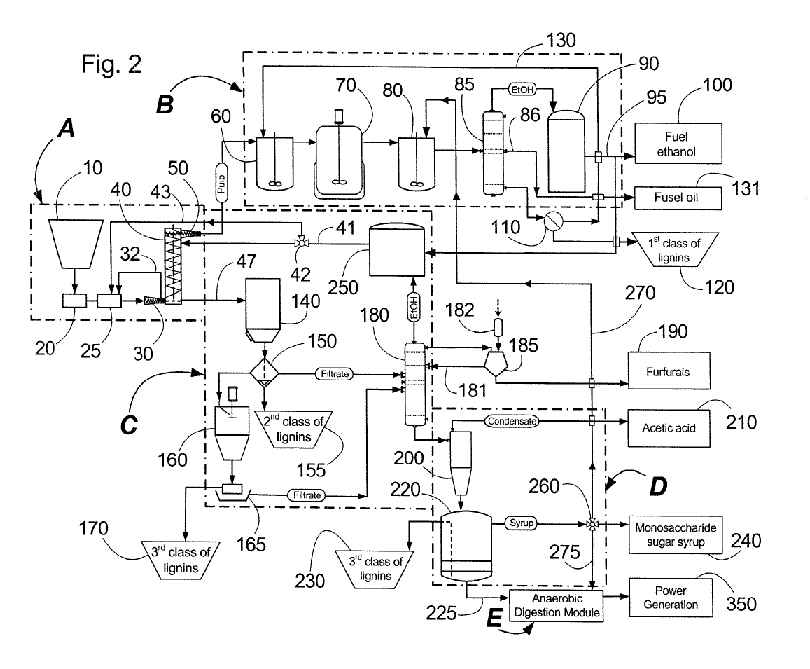

FIG. 2 illustrates exemplary modifications that are suitable for the modular lignocellulosic feedstock processing system of the present invention.

One exemplary embodiment includes provision of a pre-treatment vessel 25 for receiving therein processed lignocellulosic feedstock from the separating device 20 for pre-treatment prior to digestion and extraction by commingling and saturation with a heated digestion/extraction solvent for a suitable period of time. A suitable supply of digestion/extraction solvent may be diverted from pipeline 41 by a valve 42 and delivered to the pre-treatment vessel 25 by pipeline 43. Excess digestion/extraction solvent is squeezed from the processed and pre-treated lignocellulosic feedstock by the mechanical pressures applied by the first auger feeder 30 during transfer of the feedstock into the digestion/extraction vessel 40. The extracted digestion/extraction solvent is recyclable via pipeline 32 back to the pre-treatment vessel 25 for commingling with incoming processed lignocellulosic feedstock and fresh incoming digestion/extraction solvent delivered by pipeline 43. Such pre-treatment of the processed lignocellulosic feedstock prior to its delivery to the digestion/extraction vessel 40 will facilitate the rapid absorption of digestion/extraction solvent during the commingling and cooking process and expedite the digestion of the lignocellulosic feedstock and extraction of components therefrom.

Another exemplary embodiment illustrated in FIG. 2 provides a second diverter valve 260 interposed the sugar syrup stream discharged from the stillage processing vessel 220 in module D. In addition to directing the sugar stream to the sugar stream holding tank 240, the second diverter valve 260 is configured for controllably diverting a portion of the liquid sugar stream into a pipeline 270 for delivery into the fermentation tank 80 in module B. Such delivery of a portion of the liquid sugar stream from module D will enhance and increase the rate of fermentation in tank 80 and furthermore, will increase the volume of fuel-grade ethanol produced from the lignocellulosic feedstock delivered to module A.

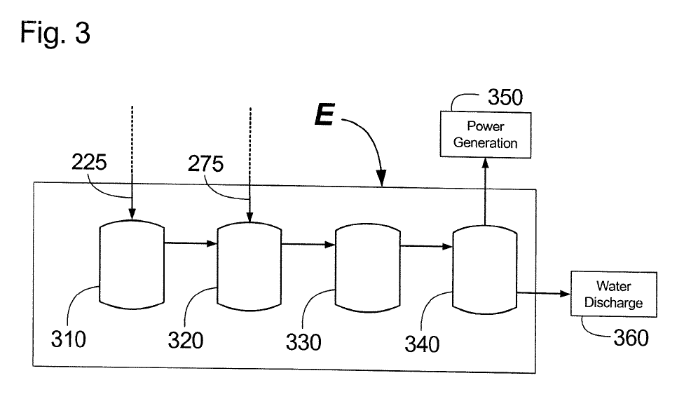

Another exemplary embodiment illustrated in FIG. 2 provides an optional fifth module E comprising an anaerobic digestion system configured to receive semi-solid/solid wastes from the stillage processing vessel 220 and optionally configured for receiving a portion of the sugar syrup stream discharged from the stillage processing vessel 220. An exemplary anaerobic digestion system comprising module E of the present invention is illustrated in FIG. 3 and generally comprises a sludge tank 310, a vessel 320 configured for containing therein biological acidification processes (referred to hereinafter as an acidification vessel), a vessel 330 configured for containing therein biological acetogenesis processes (referred to hereinafter as an acetogenesis vessel), and a vessel 340 configured for containing therein biological processes for conversion of acetic acid into biogas (referred to hereinafter as a biogas vessel). The semi-solid/solid waste materials produced in the stillage processing vessel 220 of module C are transferred by a conveyance apparatus 225 to the sludge tank 310 wherein anaerobic conditions and suitable populations of facultative anaerobic microorganisms are maintained. Enzymes produced by the facultative microorganisms hydrolyze the complex organic molecules comprising the semi-solid/solid waste materials into soluble monomers such as monosaccharides, amino acids and fatty acids. It is within the scope of the present invention to provide if so desired inocula compositions for intermixing and commingling with the semi-solid/solid wastes in the sludge tank 310 to expedite the hydrolysis processes occurring therein. Suitable hydrolyzing inocula compositions are provided with at least one Enterobacter sp. A liquid stream containing therein the hydrolyzed soluble monomers is transferred into the acidification vessel 320 wherein anaerobic conditions and a population of acidogenic bacteria are maintained. The monosaccharides, amino acids and fatty acids contained in the liquid stream received by the acidification vessel 320 are converted into volatile acids by the acidogenic bacteria. It is within the scope of the present invention to provide if so desired acidification inocula compositions configured for facilitating and expediting the production of solubilized volatile fatty acids in the acidification tank 320. Suitable acidification inocula compositions are provided with at least one of Bacillus sp., Lactobacillus sp. and Streptococcus sp. A liquid stream containing therein the solubilized volatile fatty acids is transferred into the acetogenesis vessel 330 wherein anaerobic conditions and a population of acetogenic bacteria are maintained. The volatile fatty acids are converted by the acetogenic bacteria into acetic acid, carbon dioxide, and hydrogen. It is within the scope of the present invention to provide if so desired inocula compositions configured for facilitating and expediting the production of acetic acid from the volatile fatty acids delivered in the liquid stream into in the acetogenesis vessel 330. Suitable acetification inocula compositions are provided with at least one of Acetobacter sp., Gluconobacter sp., and Clostridium sp. The acetic acid, carbon dioxide, and hydrogen are then transferred from the acetogenesis vessel 330 into the biogas vessel 340 wherein the acetic acid is converted into methane, carbon dioxide and water. The composition of the biogas produced in the biogas vessel 340 of module E will vary somewhat with the chemical composition of the lignocellulosic feedstock delivered to module A, but will typically comprise primarily methane and secondarily CO₂, and trace amounts of nitrogen gas, hydrogen, oxygen and hydrogen sulfide. It is within the scope of the present invention to provide if so desired methanogenic inocula compositions configured for facilitating and expediting the conversion of acetic acid to biogas. Suitable methanogenic inocula compositions are provided with at least one of bacteria are from the Methanobacteria sp., Methanococci sp., and Methanopyri sp. The biogas can be fed directly into a power generation system as exemplified by a gas-fired combustion turbine. Combustion of biogas converts the energy stored in the bonds of the molecules of the methane contained in the biogas into mechanical energy as it spins a turbine. The mechanical energy produced by biogas combustion, for example, in an engine or micro-turbine may spin a turbine that produces a stream of electrons or electricity. In addition, waste heat from these engines can provide heating for the facility's infrastructure and/or for steam and/or for hot water for use as desired in the other modules of the present invention.

However, a problem with anaerobic digestion of semi-solid/solid waste materials is that the first step in the process, i.e., the hydrolysis of complex organic molecules comprising the semi-solid/solid waste materials into a liquid stream containing soluble monomers such as monosaccharides, amino acids and fatty acids, is typically lengthy and variable, while the subsequent steps, i.e., acidification, acetification, and biogas production proceed relatively quickly in comparison to the first step. Consequently, such lengthy and variable hydrolysis in the first step of anaerobic may result in insufficient amounts of biogas production relative to the facility's requirements for power production and/or steam and/or hot water. Accordingly, another embodiment of the present invention, as illustrated in FIGS. 2 and 3, controllably provides a portion of the sugar syrup stream discharged from the stillage processing vessel 220 of module D, to the acidification tank 320 of module E to supplement the supply of soluble monosaccharides hydrolyzed from semi-solid/solid materials delivered to the sludge tank 310. Thus, the amount of biogas produced by module E of the present invention can be precisely manipulated and modulated by providing a second diverter 260 interposed the sugar syrup discharge line from stillage processing vessel 220, to controllably divert a portion of the sugar syrup into pipeline 275 for transfer to the acidification vessel 320.

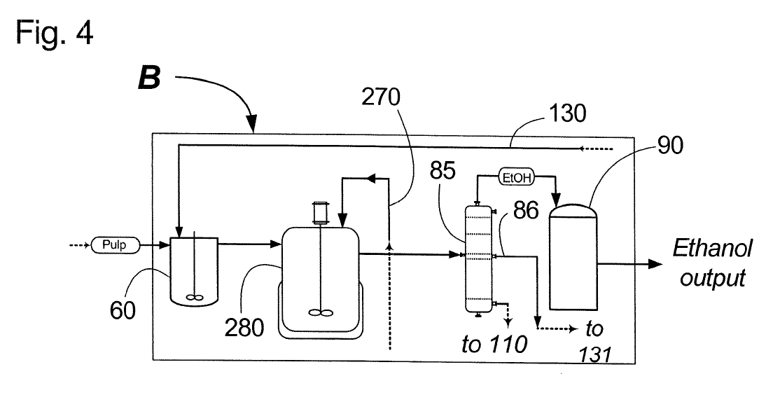

Another exemplary embodiment of the present invention is illustrated in FIG. 4 and provides an optional vessel 280 for module B, wherein vessel 280 is configured for receiving the reduced viscosity pulp from mixing vessel 60 (FIG. 2) and for concurrent i.e., co-saccharification and co-fermentation therein of the reduced-viscosity solids fractions. Those skilled in these arts will understand that such co-saccharification and co-fermentation processes are commonly referred to as “simultaneous saccharification and fermentation” (SSF) processes, and that vessel 280 (referred to hereinafter as a SSF vessel) can replace digestion vessel 70 and fermentation vessel 80 from FIG. 2. It is suitable to provide a supplementary stream of sugar syrup into the SSF vessel 280 via pipeline 270 from the second diverter valve 260 (FIGS. 2 and 4) to controllably enhance and increase the rate of fermentation in the SSF vessel 280.

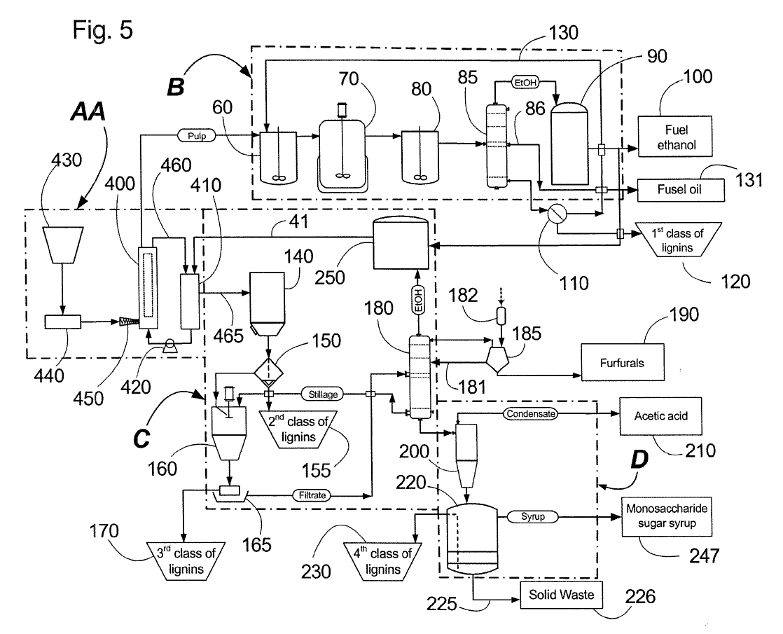

Another exemplary embodiment of the present invention is illustrated in FIG. 5 and provides an alternative first module AA, for communication and cooperation with modules B and C, wherein the alternative first module AA (FIG. 5) is configured for receiving, processing and digestion/extraction of batches of a lignocellulosic feedstock, as compared to module A which is configured for continuous inflow, processing and digestion/extraction of a lignocellulosic feedstock (FIG. 1). As shown in FIG. 5, one exemplary embodiment for batch digestion/extraction of a lignocellulosic feedstock comprises a batch digestion/extraction vessel 400 interconnected and communicating with a digestion/extraction solvent re-circulating tank 410 and a solvent pump 420. A batch of lignocellulosic feedstock is loaded into a receiving bin 430 from where it is controllably discharged into a conveyance system provided with a screening device 440 configured for removing pebbles, gravel, metal objects and other debris. The screening device 440 may be optionally configured for sizing the lignocellulosic feedstock into desired fractions. The processed lignocellulosic feedstock is then conveyed with a third auger feeder 450 into a first end of the batch digestion/extraction vessel 400. The digestion/extraction solvent re-circulating tank 410 is configured to receive a suitable digestion/extraction solvent from the digestion/extraction solvent holding tank 250 of module B via pipeline 41. The digestion/extraction solvent is pumped via solvent pump 420 into the batch digestion/extraction vessel 400 wherein it controllably commingled, intermixed and circulated through the batch of lignocellulosic feedstock contained therein. The batch digestion/extraction vessel 400 is controllably pressurized and temperature controlled to enable manipulation of pressure and temperature so that target cooking conditions are provided while the solvent is commingling and intermixing with the feedstock. Exemplary cooking conditions include pressures in the range of about 15-30 bar(g), temperatures in the range of about 120°-350° C., and pHs in the range of about 1.5-5.5. During the cooking process, lignins and lignin-related compounds contained within the commingled and impregnated lignocellulosic feedstock will be dissolved into the organic solvent resulting in the cellulosic fibrous materials adhered thereto and therewith to disassociate and to separate from each other. Those skilled in these arts will understand that in addition to the dissolution of lignins and lignin-related polymers, the cooking process will release monosaccharides, oligosaccharides and polysaccharides and other organic compounds for example acetic acid, in solute and particulate forms, from the lignocellulosic materials into the organic solvents. It is suitable to discharge the digestion/extraction solvent from the batch digestion/extraction vessel 400 through pipeline 460 during the cooking process for transfer via pipeline 460 back to the digestion/extraction solvent re-circulating tank 410 for re-circulation by the solvent pump 420 back into the batch digestion/extraction vessel 400 until the lignocellulosic feedstock is suitable digested and extracted into a solids fraction comprising a viscous pulp material comprising dissociated cellulosic fibers, and a liquids fraction, i.e., black liquor, comprising solubilized lignins and lignin-related polymers, hemicelluloses, polysaccharides, oligosaccharides, monosaccharides and other organic compounds in solute and particulate forms, from the lignocellulosic materials in the spent organic solvents. It is within the scope of the present invention to withdraw a portion of the re-circulating digestion/extraction solvent from the solvent re-circulating tank 410 via pipeline 465 for transfer to the flashing-tower 140 in module C, and to replace the withdrawn portion of re-circulating digestion/extraction solvent with fresh digestion/extraction solvent from the digestion/extraction solvent holding tank 250 of module B via pipeline 41, thereby expediting the digestion/extraction processes within the batch digestion/extraction vessel 400. After digestion/extraction of the lignocellulosic feedstock has been completed, the solids fraction comprising cellulosic fibre pulp is discharged from the batch digestion/extraction vessel 400 and conveyed to the mixing vessel 60 in module B wherein the viscosity of the solids fraction, i.e., pulp discharged from the first module AA, is controllably reduced to a selected target viscosity by commingling and intermixing with de-lignified stillage delivered via pipeline 130 then be controllably recycled from de-lignification equipment 110 of module B after which the reduced-viscosity pulp is further processed by saccharification, fermentation and refining as previously described. The black liquor is transferred from the digestion/extraction solvent re-circulating tank 410 via pipeline 465 to the flashing tower 140 in module C for precipitating lignin therefrom and further processing as previously described.

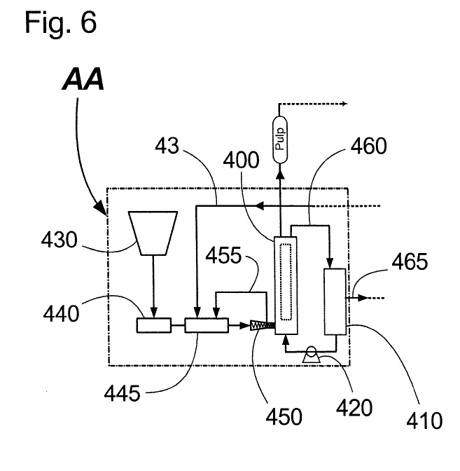

A suitable exemplary modification of the batch digestion/extraction module component of the present invention is illustrated in FIG. 6, wherein a pre-treatment vessel 445 is provided for receiving therein processed lignocellulosic feedstock from the screening device 440 for pre-treatment prior to conveyance to the batch digestion/extraction vessel 400, by commingling and saturation with a digestion/extraction solvent for a suitable period of time. A suitable supply of digestion/extraction solvent may be diverted from pipeline 41 by a valve 42 (shown in FIG. 2) and delivered to the pre-treatment vessel 445 by pipeline 43. Excess digestion/extraction solvent is squeezed from the processed and pre-treated lignocellulosic feedstock by the mechanical pressures applied by the third auger feeder 450 during transfer of the feedstock into the batch digestion/extraction vessel 400. The extracted digestion/extraction solvent is recyclable via pipeline 455 back to the pre-treatment vessel 445 for commingling with incoming processed lignocellulosic feedstock and fresh incoming digestion/extraction solvent delivered by pipeline 43. Such pre-treatment of the processed lignocellulosic feedstock prior to its delivery to the batch digestion/extraction vessel 400 will facilitate the rapid absorption of digestion/extraction solvent during the commingling and cooking process and expedite the digestion of the lignocellulosic feedstock and extraction of components therefrom.

While this invention has been described with respect to the exemplary embodiments, those skilled in these arts will understand how to modify and adapt the systems, processes and equipment configurations disclosed herein for continuously receiving and controllably commingling lignocellulosic feedstocks with counter-flowing organic solvents. Certain novel elements disclosed herein for processing a continuous incoming stream of lignocellulosic feedstocks with countercurrent flowing or alternatively, concurrent flowing organic solvents for separating the lignocellulosic materials into component parts and further processing thereof, can be modified for integration into batch systems configured for processing lignocellulosic materials. For example, the black liquors produced in batch systems may be de-lignified and then a portion of the de-lignified black liquor used to pretreat a new, fresh batch of lignocellulosic materials prior to batch organosolv cooking, while the remainder of the de-lignified black liquor is further processed into component parts as disclosed herein. Specifically, the fresh batch of lignocellulosic materials maybe controllably commingled with portions of the de-lignified black liquor for selected periods of time prior to contacting, commingling and impregnating the batch of lignocellulosic materials with suitable organic solvents. Also, it is within the scope of the present invention, to provide turbulence within a batch digestion system wherein a batch of lignocellulosic materials is cooked with organic solvents by providing pressurized flows of the organic solvents within and about the digestion vessel. It is optional to controllably remove portions of the organic solvent/black liquors from the digestion vessel during the cooking period and concurrently introduced fresh organic solvent and/or de-lignified black liquors thereby facilitating and expediting delignification of the lignocellulosic materials. It is also within the scope of the present invention to further process the de-lignified black liquors from the batch lignocellulosic digestion systems to separate and further process components parts exemplified by lignins, furfural, acetic acid, monosaccharides, oligosaccharides, and ethanol among others.

Therefore, in view of numerous changes and variations that will be apparent to persons skilled in these arts, the scope of the present invention is to be considered limited solely by the appended claims.

What is claimed is:

1-29. (canceled)

30. A modular system for organosolv fractionation of lignocellulosic feedstock into component parts and further processing of said component parts; the modular process comprising:
a first processing module configured for receiving, physically processing, and physico-chemically digesting a lignocellulose feedstock therewith a separately supplied organic solvent thereby extracting component parts therefrom said feedstock, and separating said component parts into a cellulosic solids fraction and a first liquid fraction;
a second processing module configured for receiving therein said cellulosic solids fraction and for producing therefrom at least a fuel-grade ethanol, a first class of lignin derivatives, and a de-lignified stillage;
a third processing module configured for separating the first liquid fraction into a solids fraction comprising a second class of lignin derivatives, a third class of lignin derivatives and a filtrate, for separating furfurals from said filtrate, and for recovering a portion of the organic solvent from the filtrate by distillation thereby producing a first stillage; and
a fourth processing module configured for separating the first stillage into at least an acetic acid-containing liquid fraction, a fourth class of lignin derivatives, a monosaccharide sugar syrup, and a solid waste material.

31. A modular system according to claim 30, additionally provided with a fifth processing module configured for anaerobic digestion and processing of the solid waste material separated by the fourth processing module, into at least a collectable biogas and an liquid effluent.

32. A modular system according to claim 30, wherein the first processing module comprises a plurality of equipment selected and configured for controllable and manipulable communication and cooperation for:
receiving therein a lignocellulosic feedstock;
processing the lignocellulosic feedstock by physically separating non-lignocellulosic materials therefrom;
physico-chemically digesting the processed lignocellulosic feedstock by commingling said feedstock with a separately supplied organic solvent while controllably manipulating at least the temperature and pressure therein and thereabout, thereby producing the cellulosic solids fraction and the first liquid fraction; and
separately and controllably discharging the cellulosic solids fraction and the first liquid fraction.

33. A modular system according to claim 30, wherein the first processing module is configured to continuously receive, physically process, and physico-chemically digest a lignocellulosic feedstock thereby continuously producing the cellulosic solids fraction and the first liquid fraction.

34. A modular process according to claim 30, wherein the first processing module is configured to receive, physically process, and physico-chemically digest a batch of lignocellulosic feedstock thereby producing the cellulosic solids fraction and the first liquid fraction.

35. A modular system according to claim 30, wherein the first processing module is provided with a temperature-controllable and pressure-controllable digestion vessel configured to:
receive therein the processed lignocellulosic feed stock at about a first end and to convey said feedstock therethrough to about a second end;
receive therein an organic solvent at about the second end, and to flow said organic solvent therethrough to about the first end;
discharge the cellulosic solids fraction through an outlet provided therefor approximate the second end; and
discharge the first liquid fraction through an outlet provided therefor approximate the first end.

36. A modular system according to claim 35, wherein the temperature-controllable and pressure-controllable digestion vessel is configured to:
receive therein the processed lignocellulosic feed stock at about a first end and to convey said feedstock therethrough to about a second end;
receive therein an organic solvent at about the first end, and to circulate said organic solvent therethrough and thereabout;
discharge the cellulosic solids fraction through an outlet provided therefor approximate the second end; and
discharge the first liquid fraction through an outlet provided therefor approximate the second end.

37. A modular system according to claim 35, wherein the temperature-controllable and pressure-controllable digestion vessel is configured to:
receive therein the processed lignocellulosic feed stock at about a first end and to convey said feedstock therethrough to about a second end;
receive therein an organic solvent interposed the first end and second end, and to circulate said organic solvent therethrough and thereabout;
discharge the cellulosic solids fraction through an outlet provided therefor approximate the second end; and
discharge the first liquid fraction through an outlet provided therefor approximate the first end.

38. A modular system according to claim 30, wherein the first processing module is additionally provided with equipment configured to sequentially saturate and de-saturate the processed lignocellulosic feedstock prior to physico-chemically digesting said processed lignocellulosic feedstock.

39. A modular system according to claim 30, wherein the second processing module comprises a plurality of equipment selected and configured for controllable and manipulable communication and cooperation for:
receiving therein the cellulosic solids fraction discharged from the first processing module;
reducing the viscosity of the cellulosic solids fraction;
enzymatic digestion of the reduced-viscosity cellulosic fraction thereby producing a second liquid fraction;
fermentation of the second liquid fraction thereby producing a beer therefrom;
distillation of the beer thereby producing a fuel-grade ethanol and a second stillage therefrom; and
separating a first class of lignin derivatives from said second stillage.

40. A modular system according to claim 30, wherein the de-lignified second stillage is recyclable for reducing the viscosity of the cellulosic solids fraction.

41. A modular system according to claim 30, wherein the second processing module is provided with a vessel for containing therein concurrent enzymatic digestion of the reduced-viscosity cellulosic solids fraction and fermentation of the second liquid fraction produced therefrom.

42. A modular system according to claim 30, wherein the anaerobic digestion module is provided with a plurality of equipment configured for:
receiving and biologically hydrolyzing therein the solid waste material separated by the fourth processing module thereby producing a third liquid fraction;
receiving and biologically acidifying therein the third liquid fraction thereby producing a biologically acidified liquid fraction;
receiving and biologically acetifying therein the biologically acidified liquid fraction thereby producing at least acetic acid; and
receiving the at least acetic acid and biologically producing at least a biogas and a liquid effluent therefrom.

43. A modular system according to claim 42, wherein the anaerobic digestion module is additionally configured for controllably receiving and commingling a portion of said monosaccharide sugar syrup separated in the fourth processing module with the a third liquid fraction.

44. A modular system according to claim 30, additionally provided with a sixth processing module configured for receiving, fermenting and distilling therein said monosaccharide sugar syrup, and for separating therefrom a distillate and a stillage.

45. A modular system according to claim 44, wherein said distillate comprises at least 1,3 propanediol.

46. A modular system according to claim 44, wherein said distillate comprises at least polylactic acid.

47. A first class of lignin derivatives, said first class of lignin derivatives produced by a process comprising:
a first step comprising hydrolizing a cellulosic solids material thereby producing a liquid stream containing at least soluble monosaccharides;
a second step comprising fermentation of said liquid stream containing at least soluble monosaccharides thereby producing a fermentation beer;
a third step comprising distillation of said beer to produce an ethanol and a stillage therefrom; and
a fourth step of precipitating and separating lignin derivatives from said stillage.

48. A second class of lignin derivatives, said second class of lignin derivatives produced by a process comprising:
a first step comprising fractionating a lignocellulosic feedstock into a cellulosics solids fraction and a pressurized liquids fraction, and separating said fractions;
a second step comprising depressurizing and cooling the liquids fraction thereby precipitating a plurality of lignin derivatives from said liquids fraction; and
a third step comprising separating said precipitated plurality of lignin derivatives from said liquids fraction.
